(19) United States
(12) Patent Application Publication
(10) Pub. No.: US 2026/0035757 A1
(43) Pub. Date: Feb. 5, 2026

(54) METHODS AND COMPOSITIONS FOR DETECTING MICROBIAL GROWTH IN BUILT ENVIRONMENTS

(71) Applicants: Ohio State Innovation Foundation, Columbus, OH (US); University of Notre Dame du Lac, South Bend, IN (US)

(72) Inventors: Karen Dannemiller, Columbus, OH (US); Kyle Bibby, Notre Dame, IN (US)

(21) Appl. No.: 19/285,408

(22) Filed: Jul. 30, 2025

Related U.S. Application Data
(60) Provisional application No. 63/677,706, filed on Jul. 31, 2024.

(51) Int. Cl. C12Q 1/70; C12Q 1/6895

(52) U.S. Cl. CPC C12Q 1/701; C12Q 1/6895; C12Q 2600/158

(57) ABSTRACT

Disclosed herein is a method for determining concentration or presence of at least one microbe in a built environment, the method comprising a) collecting a sample; and b) measuring concentration of at least one indicator organism, wherein the concentration of the indicator organism correlates to the concentration or presence of the at least one microbe, and further wherein the indicator organism and the microbe are different organisms. Also disclosed are systems, devices, and kits for carrying out the method.

### CROSS-REFERENCE TO RELATED APPLICATIONS

This application claims benefit of U.S. Provisional Application No. 63/677,706, filed Jul. 31, 2024, incorporated herein by reference in its entirety.

### GOVERNMENT SUPPORT CLAUSE

This invention was made with government support under R21 AI168817 awarded by the National Institutes of Health. The government has certain rights in the invention.

### BACKGROUND

During the pandemic, lack of high-quality, interpretable data led schools, hospitals, and other organizations to spend billions of dollars on questionable ventilation and air cleaning improvements. The US passed the $1.9 trillion American Rescue Plan in March 2021 that included about $200 billion for improvements in schools. However, a year later, the majority of that remained unspent, which translated to missed opportunities to improve ventilation and reduce viral transmission. A CDC report found that schools remained unsure how to improve ventilation, and that no single ventilation improvement strategy was employed by the majority of schools. Two-thirds of schools, and more in rural districts, did not replace or upgrade their HVAC system at all. If indoor ventilation approached outdoor levels, viral transmission would be reduced by orders of magnitude, which would have substantial public health impacts and could prevent future pandemic. However, there is a tradeoff with cost and energy use as buildings are responsible for 40% of our energy usage and associated greenhouse gas emissions. Unfortunately, we have no reliable quantitative measure of human microbial respiratory emissions, rendering it nearly impossible to predictably control infection risk.

What is needed in the art is the ability to evaluate the effectiveness of environmental controls in buildings to reduce viral transmission and to prioritize resources. Indicator microbial markers are widely and successfully used to track fecal contamination of water systems, but no equivalent measurement exists for respiratory emissions.

### SUMMARY

Disclosed herein is a method for determining concentration or presence of at least one microbe in a built environment, the method comprising a) collecting a sample; and b) measuring concentration of at least one indicator organism, wherein the concentration of the indicator organism correlates to the concentration or presence of the at least one microbe, and further wherein the indicator organism and the microbe are different organisms.

Also disclosed is a device for determining concentration or presence of at least one microbe in a built environment, wherein the device comprises an input unit for receiving a sample, a means for measuring presence or concentration of an indicator organism within the sample, and an output unit for indicating the presence or concentration of the at least one indicator organism, wherein the indicator organism and the microbe are different organisms.

Lastly, disclosed is a kit for determining concentration or presence of at least one microbe in a built environment, the kit comprising a) means for collecting a sample; and b) means for measuring concentration of at least one indicator organism, wherein the concentration of the indicator organism correlates to the concentration or presence of the at least one microbe, and further wherein the indicator organism and the microbe are different organisms.

### BRIEF DESCRIPTION OF THE DRAWINGS

The accompanying figures, which are incorporated in and constitute a part of this specification, illustrate several aspects of the disclosure, and together with the description, serve to explain the principles of the disclosure.

FIG. 1A-B clearly demonstrates that function analysis (B) is superior to species-specific analysis (A) for identification of moisture-damaged samples. These are principal coordinate plots where similar microbial communities cluster together. A. Dust samples from 19 sites were incubated under different moisture conditions (darker is more moisture) and only a faint moisture trend in the upper right is visible. Home-specific effects dominate any moisture signature when considering only microbial species that are present. This is why species measurements do not work and have not worked for decades. B. In the RNA (function) analysis from 9 sites, there is clear differentiation by moisture condition. Function is a better foundation for an indicator of mold growth.

FIG. 2 shows visible microbial growth in dust occurs after one week at elevated relative humidity.

FIG. 3 shows that dust is an effective matrix for surveillance of COVID-19 at the building scale.

FIG. 4 shows SARS-COV-2 variants sequenced from the dust (bars) correlates with saliva samples from individuals collected on campus (dotted lines) (p<0.05).

FIG. 5 shows dust can be an important viral monitoring tool.

FIG. 6 shows a prototype of an indoor allergen assessment system.

FIG. 7 shows a heat map of SARS-COV-2 concentrations in dust.

FIG. 8 shows SARS-COV-2 concentrations in dust vs. cases in the same building from spring 2022. There is a statistically significant association between cases and dust measures (p=0.00004). The inverse hyperbolic sine was used for data transformation to handle the highly skewed data with zeros.

### DETAILED DESCRIPTION

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

The term “antibody” is used in the broadest sense, and specifically covers monoclonal antibodies (including full length monoclonal antibodies), polyclonal antibodies, and multi-specific antibodies (e.g., bispecific antibodies). Native antibodies and immunoglobulins are usually heterotetrametric glycoproteins of about 150,000 Daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. Antibodies (Abs) exhibit binding specificity to a specific target. Antibody specificity can be assessed by comparing binding signals in cells expressing the target protein to control cells with the target gene knocked out. A highly specific antibody should show no binding activity if the target is not there. With protein antigens, the antibody molecule contacts the antigen over a broad area of its surface that is complementary to the surface recognized on the antigen. Electrostatic interactions, hydrogen bonds, van der Waals forces, and hydrophobic interactions can all contribute to binding.

The term “antimicrobial” refers to an agent that kills microorganisms or stops their growth.

The term “antibacterial” refers to an agent that is proven to kill bacteria or stops bacterial growth.

“Built environment” as used herein is any human-made, naturally-occurring or modified structure, including commercial, retail, private, governmental, educational, temporary, vehicular, and recreational structures.

As used herein, the term “buffer” refers to a solution consisting of a mixture of acid and its conjugate base, or vice versa. The solution is used as a means of keeping the pH at a nearly constant range to be used in a wide variety of chemical and biological applications.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Culture” or “cell culture” is the process by which cells are grown under controlled conditions, generally outside their natural environment. After the cells of interest have been isolated from living tissue, they can subsequently be maintained under carefully controlled conditions. These conditions vary for each cell type, but generally consist of a suitable vessel with a substrate or medium that supplies the essential nutrients (amino acids, carbohydrates, vitamins, minerals), growth factors, hormones, and gases (CO₂, O₂), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture. “Cell culture” also refers to the culturing of cells derived from multicellular eukaryotes, especially animal cells, in contrast with other types of culture that also grow cells, such as plant tissue culture, fungal culture, and microbiological culture (of microbes).

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “detect” or “detecting” refers to an output signal released for the purpose of sensing of physical phenomenon. An event or change in environment is sensed and signal output released in the form of light.

An expression “database” denotes a set of stored data that represents a collection of sequences, which in turn represent a collection of biological reference materials.

“Differentially expressed” as applied to a gene, refers to the differential production of the mRNA transcribed from the gene, or the protein product encoded by the gene. A differentially expressed gene may be overexpressed or under expressed as compared to the expression level of a normal or control cell. In one aspect, it refers to a differential that is 2.5 times, preferably 5 times, or preferably 10 times higher or lower than the expression level detected in a control sample. The term “differentially expressed” also refers to nucleotide sequences in a cell or tissue which are expressed where silent in a control cell or not expressed where expressed in a control cell.

“Expression” as used herein refers to the process by which information from a gene is used in the synthesis of a functional gene product that enables it to produce a peptide/protein end product, and ultimately affect a phenotype, as the final effect.

The term “gene” as used in this specification refers to a segment of deoxyribonucleotides (DNA) possessing the information required for synthesis of a functional biological product such as a protein or ribonucleic acid (RNA).

The term “gene expression” refers to efficient transcription and translation of genetic information contained in concerned genes.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “kit” describes a wide variety of bags, containers, carrying cases, and other portable enclosures which may be used to carry and store solid substances, liquid substances, and other accessories necessary to detect microbial growth in a built environment. Such kits and their contents along with any applicable procedures may be used to provide access to better health outcomes in accordance with the teachings of the present disclosure.

The terms “metabolite” or “metabolic compound” as used herein refers to small molecules that are generally intermediates or end products of a metabolic pathway or process.

The term “microorganism” or “microbe” mentioned herein refers to one or more forms/species of virus, bacteria or fungi.

The term “nucleic acid” as used herein means natural and synthetic DNA, RNA, oligonucleotides, oligonucleosides, and derivatives thereof. For case of discussion, such nucleic acids are at times collectively referred to herein as “constructs,” “plasmids,” or “vectors.”

As used herein, the term “polymerase chain reaction” (“PCR”) refers to a method for increasing the concentration of a segment of a target sequence in a mixture of genomic DNA without cloning or purification. This process for amplifying the target sequence typically consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times to obtain a high concentration of an amplified segment of the desired target sequence. Unless otherwise noted, PCR, as used herein, also includes variants of PCR such as allele-specific PCR, asymmetric PCR, hot-start PCR, ligation-mediated PCR, multi-plex-PCR, reverse transcription PCR, or any of the other PCR variants known to those skilled in the art.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Disclosed are the components to be used to prepare the disclosed kits as well as to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular kit is disclosed and discussed and a number of modifications that can be made to the kit components are discussed, specifically contemplated is each and every combination and permutation of the kit components and the modifications that are possible unless specifically indicated to the contrary. Thus, if a set of kit components A, B, and C are disclosed as well as a set of kit components D, E, and F and an example of a combination of the components, or, for example, a combination of kit components comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the methods and kits disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

### GENERAL DESCRIPTION OF THE INVENTION

Mold species can vary in damp areas, and secondary metabolic processes in mold can be independent of species. This invention provides an evidence-based measurement target for evaluation of mold growth in built environments based on species-independent metabolic processes. In short, products from secondary metabolic pathways of fungi are species-independent and are more effective indicators of mold growth than measurement of any specific species. The methods and kits herein are based on identification of nucleic acids, proteins, metabolites, volatile organic compounds, chemicals or a combination thereof that are differentially expressed when microbes are growing in a built environment. These nucleic acids and/or proteins can serve as targets in a quantitative microbial growth measurement method. The targets can be detected in a variety of ways discussed herein.

Disclosed herein is a metatranscriptomic pipeline to analyze gene expression of microbial communities in house dust. To do this, first the challenges associated with high levels of RNases in dust and bioinformatic processing were overcome. Interestingly, these patterns in gene expression have important implications. For instance, many allergens are present in secondary metabolic pathways. This results in increased gene expression of allergenic proteins at higher moisture levels.

Fortunately, many secondary metabolic pathways in filamentous fungi have been elucidated because of interest in drug development. Many of these processes are consistent throughout the fungal kingdom, such as those related to germination and growth, and thus are independent of the presence of any given species.

The invention provides a quantitative measurement technique that avoids subjectivity in microbial growth assessment and more robust results, which was a long-felt need. The lack of such a test is partially due to the complex nature of these indoor exposures. Each home contains a unique and diverse microbial community that varies based on surface type, as well as a complex mixture of chemicals. Microbial species in a home can number in the hundreds to thousands. The present invention provides methods related to indicators inherently associated with the presence of excess moisture and microbial growth. This invention takes advantage of the advent of high-throughput DNA/RNA sequencing, which presents an important opportunity to vastly improve exposure assessment. Previously, the use of culture-based methods to study microbial communities could only reveal a small fraction of these organisms present. For instance, only 17% of fungal species are culturable, and still others might not be detected such as viable-but-not-culturable spores, non-propagating fungal fragments, and species that grow slowly. In contrast, high-throughput (or next-generation) DNA sequencing can identify all species present without a priori selection and can indicate quantitative values when coupled with quantitative polymerase chain reaction (qPCR). Additionally, the use of RNA sequencing reveals microbial function within an entire community. The use of this cutting-edge technology on environmental samples represents an underutilized opportunity to reveal answers to fundamental questions about the microbial processes that occur in damp buildings.

### Surfaces for Collection

Various types of air filters and dust-accumulating components may be used in a building environment to assess indoor air quality. These include filters integrated into heating, ventilation, and air conditioning (HVAC) systems, such as pre-filters, panel filters, pleated filters, electrostatic filters, and high-efficiency particulate air (HEPA) filters. Pre-filters and panel filters are often used to capture larger particulates, while pleated filters provide increased surface area for particulate collection. HEPA filters are commonly used in both commercial and residential systems to capture fine particulate matter with high efficiency, typically ≥99.97% of particles≥0.3 microns. Activated carbon filters, which are often used in conjunction with particulate filters, can adsorb gaseous pollutants and volatile organic compounds (VOCs), providing a means of monitoring chemical as well as particulate contaminants.

More advanced systems may include bag filters, rigid cell filters, and V-bank filters, which are configured to handle higher airflow volumes in commercial or industrial applications. Electrostatic filters and electrostatic precipitators utilize electric charges to capture dust particles and are commonly found in both portable and centralized systems. Ultraviolet (UV) filtration systems with integrated dust traps may also be present, particularly in healthcare or laboratory settings, allowing for both microbial inactivation and particulate analysis.

Dust and airborne particles may also be collected from components such as return air grilles and filters, supply air vents, and exhaust fan filters located in bathrooms, kitchens, or laboratories. Make-up air units, which bring outdoor air into a building, typically contain filters that trap particulates from both interior and exterior sources. In residential and office environments, additional sampling sources include portable HEPA air purifiers, window-mounted air conditioning unit filters, and ductless mini-split system filters, which often include washable mesh filters. Fan coil units and radiators may also have built-in or adjacent filters that collect particulate matter over time.

Other useful sampling surfaces include non-traditional or passive dust collectors such as carpet and rug fibers, ceiling tile surfaces in drop ceilings, and fabric-based air diffusers. While not designed for active filtration, these materials can accumulate airborne dust and particulates over time and are often used in microbial and chemical exposure studies. Return duct interiors and furnace filters in forced-air systems also provide accessible locations where airborne particulates may settle or become trapped. In certain buildings, especially those with sensitive air quality requirements, multiple filter types may be layered or used in tandem to provide comprehensive environmental monitoring potential.

### Methods

In one aspect, a method is provided for determining the concentration or presence of at least one microbe in a built environment. The method comprises: (a) collecting a sample from a surface, air handling system, or other dust-accumulating site within the environment; and (b) measuring the concentration or presence of at least one indicator organism in the sample.

As used herein, an “indicator organism” refers to a biological entity, such as a bacteriophage, bacteria, fungus, or other microbial species, that co-occurs, correlates, or otherwise associates with the presence of a distinct target microbe. The indicator organism and the target microbe are not the same species or strain. The method relies on a correlation between the abundance of the indicator organism and the presence or level of the target microbe. This may be based on shared ecological niches, known cohabitation patterns, or epidemiological data.

The built environment may include, but is not limited to, residential buildings, office spaces, hospitals, industrial facilities, schools, or mobile environments such as vehicles, including buses, trains, airplanes, or ships. These environments often contain complex air circulation systems, where microbes and dust particles may accumulate and be recirculated.

The sample is preferably collected from areas likely to accumulate airborne dust or bioaerosols, including but not limited to ventilation systems, HVAC (heating, ventilation, and air conditioning) units, exhaust fans, air purifiers, filters, ducts, vents, or vacuum systems. Dust and filter debris collected from such systems may contain a historical record of microbial and chemical exposures over time.

In some embodiments, the microbial target comprises one or more viruses, bacteria, or fungi. Non-limiting examples include respiratory pathogens such as Staphylococcus aureus, Mycobacterium tuberculosis, Influenza A virus, Aspergillus fumigatus, or SARS-COV-2. The indicator organism may be a bacteriophage that infects a common cohabiting microbe, or a non-pathogenic bacterial species that correlates with the presence of the target microbe in shared environments.

In certain embodiments, the concentration of a single indicator organism may be used to infer the presence or quantity of two or more distinct microbes. In other embodiments, more than one indicator organism may be measured. Optionally, more than one molecular marker may be quantified from a single indicator organism to improve specificity or predictive accuracy. For example, 2, 3, 4, 5, 6, 7, 8, 9, or 10 or more microbes can be indicated. 2, 3, 4, 5, 6, 7, 8, 9, or 10 or more indicator organisms can be measured.

Measurement of the indicator organism may be accomplished by detecting one or more biological markers associated with the organism. A marker may be a molecular component, such as a nucleic acid, protein, metabolite, or small molecule. For example, the presence of a specific DNA or RNA sequence unique to the indicator organism may be used to quantify its abundance using methods such as quantitative PCR, digital PCR, next-generation sequencing, or isothermal amplification. Alternatively, antibodies or aptamers may be used to detect proteins expressed by the indicator organism. Metabolomic analysis may also be employed to detect volatile organic compounds (VOCs) or other microbial metabolites. 2, 3, 4, 5, 6, 7, 8, 9, or 10 or more molecular markers from each indicator organism can be detected.

The methods described herein may optionally be used to infer microbial presence in a human subject who has previously occupied or interacted with the built environment. For example, the presence of an indicator organism on a filter surface may reflect recent exposure of the environment—and potentially a resident or worker—to a target microbe.

Disclosed herein is a method of inhibiting or reducing microbial growth in a built environment, wherein a built environment is a natural or man-made structure, or building wherein people live or work for example a house, laboratory, hospital, manufacturing plant, airport, airplane, spacecraft, school, and office. Increase in the humidity and decrease in ventilation of such a built environment can support the growth of microbes such as bacteria and fungi, especially mold.

### Mold

Mold is a type of fungi and can be broadly classified into three types: Allergenic, Pathogenic and Toxigenic. Allergenic mold species are those that trigger allergic reactions such as asthma. Some examples for allergenic mold species are Chaetomium, Alternaria, Ulocladium, Serpula, Mucor, Aureobasidium and Penicillium. Pathogenic mold species cause disease in immunocompromised individuals. In some embodiments, the pathogenic mold species is Aspergillus. Toxigenic mold species create and produce their own toxins which can lead to health problems that are sometimes lethal. In some embodiments, the toxigenic mold species are Stachybotrys or black mold and Trichoderma. It is most common for mold to grow in houses damaged by flooding and large water leaks, and with poor air quality. Stachybotrys is associated with sick building syndrome. This mold can be dangerous and needs to be removed only by a licensed remediation specialist who can treat the built environment affected by mold by eliminating excess moisture.

### Humidity

In some examples, the built environment can have an equilibrium relative humidity (ERH) of 30%-100%. As further disclosed herein ERH is the relative humidity of the atmosphere at a particular temperature at which a material neither gains nor loses moisture. In some embodiment of the disclosure herein, the ERH can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any amount below or in-between these values. Further disclosed herein is the first step in the method of inhibiting or reducing microbial growth is identifying the microbial growth by detecting one or more gene(s) or product(s) thereof associated with the fungal growth processes including but not limited to sporulation, hyphal growth and conidium formation and other fungal growth-related functional processes in at least one sample collected from the built environment. In some embodiments the sample can be a dust sample, a surface sample, an air sample, and/or a combination of environmental samples. Sporulation is the process by which a vegetative cell undergoes a developmental change to form a metabolically inactive spore, or endospore in the scarcity of nutrition and optimal growth conditions.

### Treatment

Once the gene expression is identified to indicate microbial growth a treatment can be applied to inhibit the microbial growth. In some embodiments, the microbial growth inhibition techniques comprise the use of a dehumidifier, an exhaust fan, an anti-microbial compound, a hydrophobic paint, or a combination thereof. In some embodiments, the treatment can be administered hourly, every 2, 3, 4, 5, 6, 7, 8, 9, 10, 11, 12, 13, 14, 15, 16, 17, 18, 19, 20, 21, 22, 23 hours, daily once, twice or three times weekly, monthly for up to 1, 2, 3 week(s), 1, 2, 3, 4, 5, 6, 7, 8, 9, 10, 11 month(s), 1, 2, or 3 years. In some embodiments the anti-microbial treatment can be anti-fungal and/or antibacterial.

### Devices

In another aspect, a device is provided for determining the concentration or presence of a microbe in a built environment based on measurement of an indicator organism. The device comprises: (a) an input unit configured to receive or process a sample; (b) a detection module or sensor configured to measure the presence or concentration of the indicator organism within the sample; and (c) an output unit configured to display or report the measured concentration or presence of the indicator organism.

The device may further comprise a processor or computer-readable means for calculating the correlation between the indicator organism and the target microbe. This may include software or firmware encoded with a correlation algorithm or model trained on empirical or published data. The device may display a qualitative or quantitative assessment of microbial burden based on one or more measured indicators.

The device may be permanently mounted or temporarily deployed in a built environment, such as a residential or commercial HVAC system, an industrial air filter unit, or a mobile environmental sampler. The device may also be handheld or portable. Samples may be collected directly from airflow (e.g., air sampling cartridges or filters), or retrieved from passive dust collectors such as vent surfaces, dust traps, or removable HVAC filters.

Like the methods described above, the device may be configured to detect nucleic acids, proteins, or other molecular markers. It may analyze more than one indicator or more than one marker from a single indicator to enhance accuracy. The device may be particularly configured to detect bacteriophages, which can act as highly specific and sensitive indicators of bacterial communities.

### Kits

In a further aspect, a kit is provided for determining the presence or concentration of a microbe in a built environment. The kit can comprise: (a) means for collecting a sample (e.g., swabs, filter removal tools, vacuum heads, or dust traps); and (b) means for measuring the concentration of at least one indicator organism.

The measurement means may include reagents for nucleic acid extraction and amplification, immunoassay reagents, lateral flow devices, or pre-loaded sequencing or hybridization chips. The kit may optionally include control reagents, calibrators, or software for analysis and data interpretation.

The disclosed methods, devices, and kits may be used for environmental monitoring, epidemiological surveillance, infection control in healthcare settings, indoor air quality assessment, bioterrorism detection, or public health studies evaluating microbe transmission and colonization patterns in shared spaces.

As disclosed herein, the kit can comprise a sample collection device. In some embodiments, the sample collection device is selected from a group comprising of a swab, a brush, sterile tubes with lids, vacuum cleaner with a sterile collection bag, a canister, a zip-top bag, or a combination thereof for the sterile collection of samples, wherein a sample is a dust sample, a surface sample, an air sample, and/or a combination of environmental samples.

Also disclosed herein, the kit further can comprise a glass chamber, for incubating the soil samples collected from the built environment and a salt solution or distilled water to maintain relative humidity along with a AquaLab™ dew point water activity meter to measure the relative humidity of the sample. As disclosed herein, the kit can further comprise a sample resuspension buffer, a lysis buffer, a wash buffer, a phenol, and chloroform for extraction of nucleic acids and proteins. Wherein, during the phenol-chloroform extraction, a mixture of phenol, chloroform, and isoamyl alcohol is added to samples to promote the partitioning of proteins, lipids and debris into an organic phase, leaving the DNA in the aqueous phase. Further enclosed in the kit are one or more control sample(s), a nucleic acid or protein detection probe, DNA or RNA polymerase and thermocycler or a lateral flow chromatography device. In some embodiments, the nucleic acid detection probe is a pair of forward and reverse primers and the expression of the one or more gene(s) is identified and quantified by quantitative polymerase chain reaction (qPCR).

In some embodiments, the protein detection probe can be an antibody, and the one or more product(s) thereof is detected in a whole protein lysate obtained from the at least one sample by lateral flow chromatography wherein the lateral flow chromatography device comprises of a protein lysate loading well, protein detection probe bound to a nitrocellulose membrane and a sample running buffer. In some embodiments, decanted sample resuspension buffer can be collected after resuspending the sample and loaded on the lateral flow chromatography device. As disclosed herein the qPCR gene expression and protein density results can be read and quantified via a smart phone-based application. Furthermore, the kit comprises components for comparing the expression of the one or more gene(s) to a control with a threshold value, database value, normalized value, relative value, validated value, or a combination thereof. The increase in the gene(s) expression or the quantity of the product(s) in collected samples as compared to the levels in controls indicates microbial growth.

### EXAMPLES: Respiratory Emission Specific Indicators Present in the IndooR Environment (RESIPIRE)

If indoor ventilation approached outdoor levels, viral transmission would be reduced by orders of magnitude, but there is a tradeoff with cost and energy use as buildings are responsible for 40% of our energy usage and associated greenhouse gas emissions. Unfortunately, there is currently no reliable quantitative measure of human microbial respiratory emissions. Disclosed herein is an indicator to quantify the presence of microbes from human respiratory emissions in indoor spaces, similar to indicators used in water systems for over 150 years. The indicator is a microbe in high concentration in human emissions that mirrors the fate of both known and unknown respiratory pathogens. This information can be utilized to optimize ventilation to reduce transmission of pathogens while minimizing energy use.

### Example 1: Fungal Function in Our Homes

Exposure of asthmatics to mold in homes costs $22.4 billion per year in the United States. These damp and water-damaged homes with visible mold growth have an established negative impact on human health. However, the precise causal mechanism remains elusive. No such species has yet been causally identified in the literature after decades of work, and there is currently no validated quantitative test in use. One marker, (1-3)-β-D-glucan, is a cell wall component that is commonly used as a fungal indicator. However, this marker is not consistently associated with health outcomes, possibly because it is not associated with growth and it is not present in all species, including some groups like Cryptococcus spp. associated with health outcomes. All quantitative tests have had weaker associations with health outcomes when compared to simple visual inspection and detection of moldy odor.

Despite the assumptions regarding specific harmful species, it is instead microbial function/growth that is fundamentally important. In fact, the moisture-dependent processes of germination and growth result in increased allergen release. In human airways, fungal growth specifically (not just presence) activates eosinophils, which releases major basic protein (MBP) and cause airway inflammation in diseases such as asthma. Fungal function/growth in the indoor environment can release similar material that causes the same effects upon inhalation.

Many fungal metabolic processes are consistent throughout the fungal kingdom, such as those related to germination and growth, and thus are independent of the presence of any given species. This makes products of these secondary metabolic pathways prime measurement targets as universal indicators of fungal growth. For instance, some genes such as sepA, hypA, and podB-podD are associated with fungal growth in the cell because they are involved in spore germination and hyphal extension.

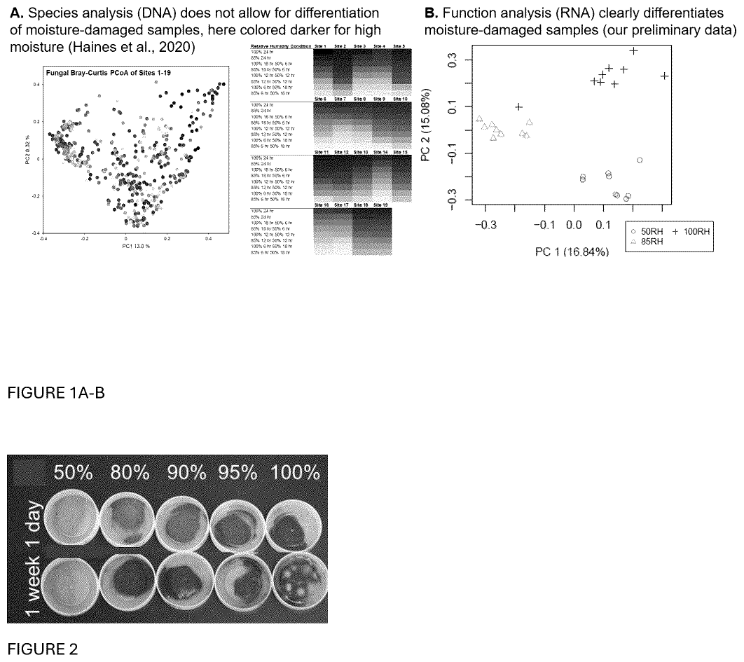

Moisture does change fungal species that are present, but that site-specific effects dominate any moisture signature in the overall community because species vary dramatically between homes (FIG. 1). However, even different fungal species in different fungal communities participate in the same cellular processes related to growth and other metabolic processes. These are the indicators that can be targeted to measure mold growth.

Results can be used to change the way mold is measured in housing by developing markers of mold growth in homes to help address health concerns, especially of the 8% of the population with asthma. Knowledge of which patients are exposed to mold can be integrated into asthma management and prevention efforts. For instance, remediation efforts in asthma homecare programs for different allergens can ultimately be targeted to patients that are both sensitized and exposed, while mold remediation is important for any patient with respiratory concerns. Results also have important implications for understanding metabolic processes in fungi. For instance, recent results showed that microbes are still metabolically active at even as low as 50% relative humidity. While these cells may not undergo reproduction, they are still producing RNA and are capable of growing upon exposure to moisture.

### Example 2: Predictive Modeling of Fungal Growth in Indoor Spaces

Moisture is critical to support microbial growth in the indoor environment. It is generally the limiting factor for growth because shed human skin and other organic materials provide sufficient nutrients in dust. It has been shown that carbon, nitrogen, phosphorous, and other micronutrients are present in excess to support growth. In fact, it has been demonstrated that microbial growth in house dust can make a substantial contribution to human exposure to microbes under elevated relative humidity (RH) (>80%).

Moisture in the indoor environment has important microbial effects that are not yet characterized. Even short periods of moisture can result in growth, and moisture in homes changes microbial communities. It has been established that in indoor environments, microbial growth in house dust will occur after one week starting at 80% relative humidity. In fact, this growth in dust is visible at 100% relative humidity (FIG. 2). Growth also likely occurs at lower relative humidity but may require a longer incubation period. Fungi grow more readily than bacteria, and after one week the concentration in dust can increase by 27.5 times the original concentration. Microbial growth rates were calculated on the order of 10⁴-10⁵ spore equivalents or bacterial genomes per mg dust per day at 85-100% relative humidity³⁰. Increased moisture dramatically changes the composition of the microbial community as well. Dust at higher relative humidity can contain more species known to produce allergens, which are also commonly found in buildings with known moisture problems.

A predictive model has been used to quantify this growth based on moisture. Using this model, we can provide critical, accurate, and rapid information about where mold growth occurs and when remediation may be necessary. This can include temporary ventilation system failure that would lead to elevated moisture. The results indicate when remediation is necessary, and can also provide potential targets for indicators of problems.

Collectively, this work contributes to development of guidelines for buildings related to prevention of mold growth. It is proposed NOT to measure mold growth on drywall, but instead focus on mold growth in the dust, which is the main source of exposure. This work intentionally focuses on fungal growth in dust based on an analogy to studies of childhood lead poisoning due to exposure in homes. One major breakthrough in understanding how lead paint impacts children came from a focus on house dust and wipe sampling. Prior to this work, measurements were generally taken from walls and other areas that may not have directly impacted exposure. Finally, directly sampling the source of lead exposure (dust) resulted in finding the associations to blood lead levels in children. Lead could then be appropriately measured and regulated.

### Example 3: Quantify Shedding of Respiratory Microbes into the Environment

Disclosed herein is a method of monitoring disease frequency due to the emergence of new viral variants and also individuals who do not respond to vaccines or refuse vaccination. Wastewater monitoring is effective for both population and building level monitoring, but samples are difficult to collect, require substantial processing prior to analysis, and not everyone sheds the virus in feces. Sewer flow may also not allow targeted sampling of some areas. We need to evaluate other methods for continued surveillance of disease frequency and viral variants in high-risk buildings such as dormitories, prisons, and congregate care facilities.

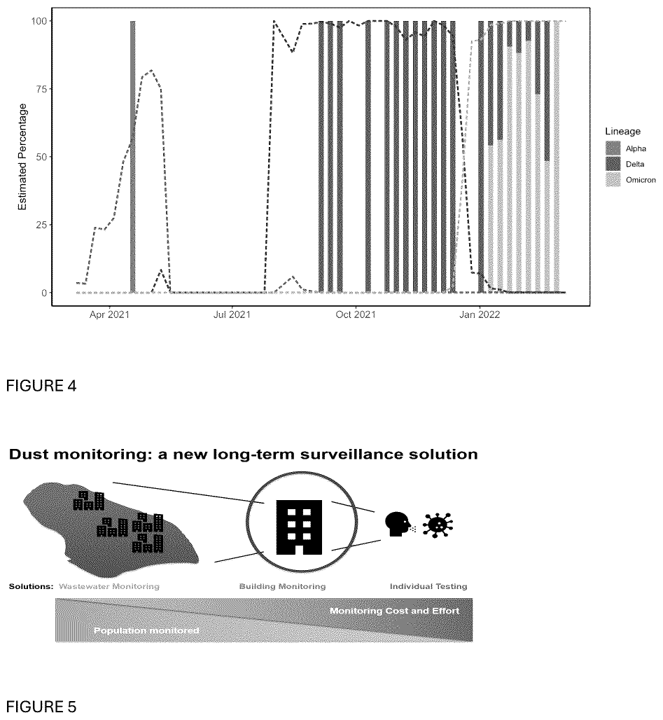

Vacuumed dust can be used as a new environmental surveillance tool to monitor respiratory viral outbreaks. Among the three methods used, bulk dust samples were 89% positive compared to 55% of surface swabs and 21% of passive air samples. Using droplet digital Polymerase Chain Reaction (ddPCR) increased the dust positivity rate to 97%, and recent work has increased this even further. A dust sample extracted prior to the pandemic and all other dust samples from homes of non-infected individuals have tested negative. We can sequence COVID-19 variants (FIG. 4). The RNA in the dust samples also persisted over time. Over four weeks, there was no measurable decay in RNA quantity (R2=0.009, P=0.47). Fortunately, this indicates that dust samples do not need to be stored in cold conditions prior to analysis, and also showed that the viability decays rapidly, much faster than RNA.

### Example 4: Characterization of Viral Shedding in Buildings at the Population Level

50 buildings per week were monitored on the Ohio State University campus and several local schools and daycares to monitor for SARS-COV-2 and influenza, as well as other viruses. Dust is a convenient matrix for monitoring of viral disease by comparing measurements of SARS-COV-2 in building dust to individual-level COVID-19 testing data. This technique is a useful tool to continue to monitor disease frequency and track variants when individual testing is no longer routinely used on a large scale. The large-scale individual testing provides a unique opportunity to understand viral dynamics in buildings that are not available in the long-term. This method can be validated, with new bioinformatics approaches developed for identifying variants, and gain new fundamental insights into viral presence in the built environment.

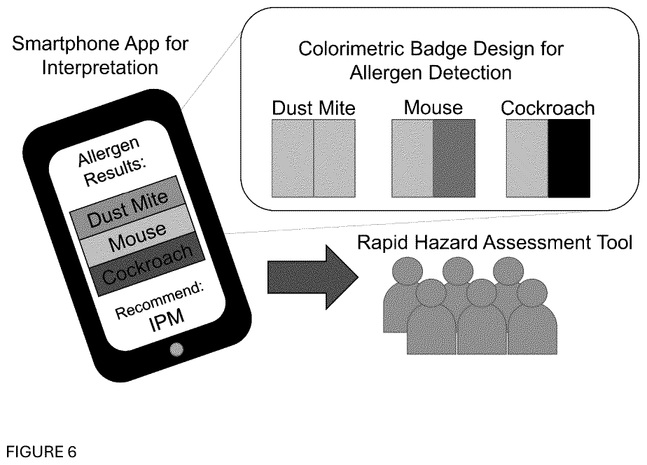

Viral disease prevention and mitigation can be directed in the future based on environmental data. This PCR-based analysis uses instrumentation that is already widely deployed for individual testing. Many facilities that are currently in use for individual level testing using PCR-based methods could be easily employed for building level monitoring using dust samples. Additionally, dust collection (vacuuming) is already being done in many buildings, and the samples are stable for long periods of time to allow for sample transport. Finally, collection, extraction and processing is easier than for wastewater samples and does not require preconcentration steps. The data analysis methods and decision-making guidance developed for wastewater monitoring can also be applied to dust monitoring data and requires interdisciplinary input. Therefore, the data from this method can quickly be utilized in combination with existing infrastructure to implement this strategy on a large scale (FIG. 5).

### Example 5: Improved Sensing of Indoor Contaminants to Empower Individuals

The environment in our home is an important part of health that is generally overlooked. Improved understanding of the biology indoors can bring personalized medicine approaches to fields such as asthma management. For instance, the National Institutes of Health in 2020 called for integrating measurements of indoor allergens into asthma care, although techniques to complete the necessary measurements are not available due to a lack of understanding of the biology of homes. The development of indoor sensors supports integration of exposure reduction methods into disease management and prevention. A monitor can be used in the home of asthmatic children (FIG. 6).

It has been found that phthalate degradation can occur in house dust through both chemical and microbial processes. This is only one of hundreds of chemicals that could be impacted by microbes indoors. Other indoor environments, such as aircraft and submarines, can be impacted by these degradation processes, and these processes may be initiated in the dust. It has also been established that RNA expression can occur even at 50% RH (typical indoor level).

This work can fundamentally change how we measure mold indoors, which currently costs $22.4 billion per year from exposure to asthmatics alone. An evidence-based measurement technique is sorely lacking, and environmental practitioners are currently forced to choose between using unvalidated quantitative tests or subjective/qualitative tests to assess for mold in homes. The possibility of enhanced measurement has important implications for asthmatics and the general public who are affected by mold. Currently, many of the affected homes are also disproportionately located in low-income areas and house vulnerable populations who would greatly benefit from this detection technology. This analysis becomes increasingly important as climate change causes an increase in natural disaster and flooding events associated with mold growth. Measurement can inform decision-making in the indoor environment, where high-cost tradeoffs between energy, indoor environmental quality, and health are considered. This work allows remediation specialists to use a more robust, quantitative tool in their assessments. Improved identification of damaged homes can result in cost savings and improved health for residents, and can be integrated into disease management such as through asthma homecare programs.

This invention can be used to retrieve building-level resolution testing data to monitor for viral outbreaks in future epidemics/pandemics. It can be used for ongoing viral surveillance for bioterrorism activities or to detect the presence of future pandemic viruses as they reach new areas. It can be used to provide early detection of viral illness in many congregate settings, such as nursing homes and schools. Overall, this work lays the groundwork for widespread implementation to support the fight against a wide range of respiratory viral illnesses.

### Example 6: Research Approach

Currently, no test exists using an indicator organism to confirm that ventilation is adequate in a given building to reduce viral transmission. Right now, the best analogy is that it is like we are trying to control temperature in buildings without a thermometer. Development of a novel indicator for the presence of human respiratory emissions can transform capability to understand and quantify infection risk for a wide range of illnesses in any space and adjust ventilation accordingly to reduce transmission.

Respiratory Viruses are Emitted into Air in Buildings and Deposited into Dust

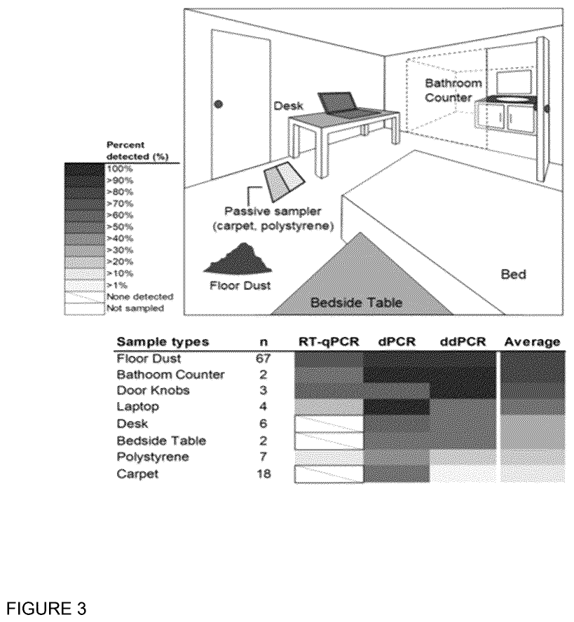

It has been found that bulk dust had the highest concentration of viral RNA and the highest number of consistent positive detects (FIG. 3). The virus can be deposited into dust via respiratory droplets. Among the three methods used, bulk dust samples were 89% positive compared to 55% of surface swabs and 21% of passive air samples. Using droplet digital Polymerase Chain Reaction (ddPCR) increased the dust positivity rate to 97%. A dust sample extracted prior to the pandemic and all other dust samples from homes of non-infected individuals have tested negative.

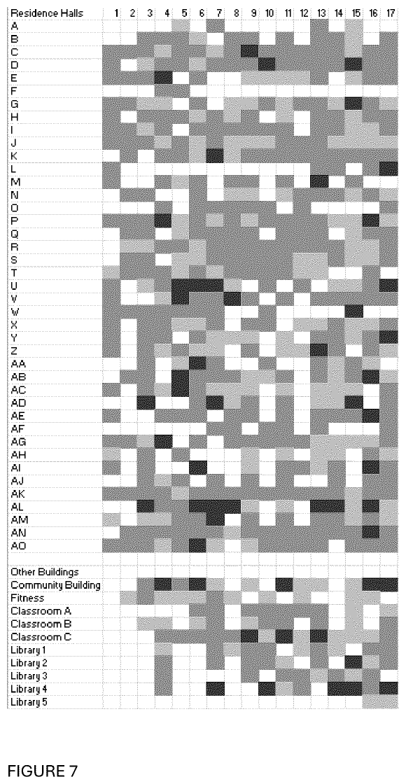

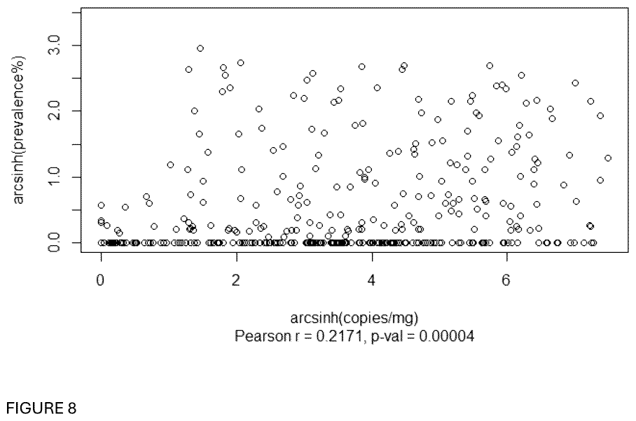

Furthermore, dust has been sampled from over 50 buildings on the Ohio State University campus and 6 Columbus area schools on a weekly basis (FIG. 7). We have over 3500 samples processed. The data correlate well with known cases (FIG. 8, p=0.00004). We have demonstrated that the virus is unlikely to remain infective in dust¹⁹ and recently had a manuscript accepted demonstrating that we can sequence the variants from the dust and they correlate with viruses sequenced from human saliva (FIG. 4). Thus, dust acts as a long-term cumulative proxy for viruses that were recently in the air.

### Microbial Indicators are Successfully Used in Water System to Indicate Pathogen Risk

Fecal indicators have been used in water systems for over 150 years for risk characterization, source attribution, and impact evaluation. These organisms are used as proxies to indicate the risk of the presence of fecal-oral pathogens that are impractical to monitor routinely. Recently, the next-generation of indicator organisms has included cross-assembly phage (crAssphage) and pepper mild mottle virus (PMMoV) as viral water quality indicators. These organisms are highly abundant in sewage, universally present in the human population, and correlate well with viral pathogens compared to use of bacterial or individual pathogens These properties set the stage for integration into Quantitative Microbial Risk Assessment44 and water quality standards. However, until now, there was not a similar indicator for use in air/dust.

What is needed is a novel innovative approach to evaluate the effectiveness of environmental controls such as ventilation in curtailing viral transmission to produce evidence-based recommendations for ventilation requirements to reduce viral transmission in buildings to prioritize resources. A microbial indicator in dust can be used to monitoring human respiratory emissions in air in buildings.

The indicator is a marker for a microbe in high concentration in human emissions that mirrors the fate of both known and unknown respiratory pathogens. It can better account for use of filter-based air cleaning technologies over other measures such as carbon dioxide. The indicator can be widely implemented in buildings to inform ventilation control and standards to reduce respiratory illness.

Indoor dust samples, indoor air samples, and human saliva samples are screened for bacteriophage targets in consistent and high abundance. Their distribution indoors is then calculated. Common indicator organisms such as crAssphage and/or PMMOV can be among the most viable targets, in addition commensal organisms in high concentration.

100 individuals from around the United States are recruited to provide a saliva sample, a dust sample from their home, and a mouth swab from any mammalian pets. Recruitment protocol ensures diversity of geography, age, and socioeconomic background by posting widely and asking about these criteria. Individuals receive a kit in the mail, and collect and ship the samples back to us with a prepaid shipping label. These samples undergo untargeted sequencing of both DNA and RNA and the most abundant fragments of DNA and RNA that are present in the samples that originate from viruses or bacteria.

We seek to determine how indicator organisms are exhaled into the air and then deposited into dust, which our work has shown works as a long-term cumulative sample of respiratory viral emissions. 20 individuals are used for testing in chambers. A saliva sample is collected at the beginning, and an air sample during, and a dust sample after from a well-characterized environmental chamber where people sit for 3 hours. Following sitting, we will simulate talking for 1 hour, with air/dust sampling, and then simulated coughing (30 second cough, 30 second rest) for 15 minutes, with air/dust sampling. qPCR assays are used to measure the top targets found in Task 1.1 to characterize their deposition in Task 1.2.

### Objective 2: Validate the Marker Against COVID-19 Transmissions in a Unique Dust Dataset.

Measures of the target indicators, SARS-COV-2, COVID-19 cases, building occupancy, information, and ventilation in buildings are compared to determine which indicator organism is the most effective at correlating with human emissions and disease transmission.

The dataset and 3500+ banked dust samples from buildings on the Ohio State University campus are used to measure the marker and find associations with transmission rates. Dust serves as a proxy for organisms that were recently (within recent weeks) in the air. Dust is also easier to sample and recover biomass. Data indicates that CrAssphage and PMMoV are measurable in building dust.

The pandemic provided an unprecedented scenario for collection of real-time infection data for a respiratory virus in a large population. Here, this irreplaceable data is coupled with detailed ventilation information and environmental deposition to yield quantitative insights into the development of a novel indicator of respiratory contamination of the indoor environment. Residence halls are an optimal space to study transmission because we can combine detailed information on varied ventilation systems with weekly infection information among individuals sharing indoor space among the approximately 10,000 residential students on OSU's campus.

In situ ventilation and infiltration rates are measured using standard tracer gas methods (both steady state and decay tests). We can measure real-time ventilation and infiltration rates for a variety of student housing unit types during vacancy periods. Literature values are used to estimate standard values for other important factors (rates of door usage, etc.).

Testing is repeated in different rooms for each configuration until a sample size sufficient to provide acceptable confidence in conclusions is met. The ventilation measurement team work in close collaboration with the statistics team to identify when a sufficient number of points have been generated.

Case data consists of serial PCR screening tests for the population (1 year) and case reporting data (3 years). All case data is currently stored in a secure analytic environment. Available surveillance data includes variables that were used for real-time case management and policy decisions during multiple variant waves and varying vaccination status.

Two approaches are used which include 1) pairwise analysis to estimate secondary attack risk within rooms, floors, and dormitories, also integrating ventilation data, together with 2) population level analysis using a modeling and inference framework for using repeat testing data to estimate infection prevalence and incidence. Both individual and population approaches use existing data from large scale SARS-COV-2 serial testing conducted at OSU (e.g., weekly testing conducted on all residential students during the 2020-2021 academic year).

Development of indicator marker for human respiratory emissions in indoor spaces can create a new paradigm to reduce transmission of viral illness. Such a measure can be implemented widely to 1) quantify infection risk from respiratory pathogens in indoor spaces in a range of scenarios, 2) be integrated into sensor technologies that alter ventilation rates to optimize tradeoffs between energy usage and infection risk, and 3) increase preparedness for future epidemics/pandemics. The findings have the potential for widespread and rapid implementation.

This PCR-based analysis uses instrumentation that is already widely deployed for individual testing. Many facilities that are currently in use for individual level testing using PCR-based methods could be easily employed for building level monitoring using dust samples. Additionally, dust collection (vacuuming) is already being done in many buildings, and the samples are stable for long periods of time to allow for sample transport. Finally, collection, extraction and processing is easier than for wastewater samples and does not require preconcentration steps. The data analysis methods and decision-making guidance developed for wastewater monitoring can also be applied to dust monitoring data (CDC 2021). Therefore, the data from this study can be utilized in combination with existing infrastructure to implement this strategy on a large scale.

This monitoring has a wide range of applications. It can be used to retrieve building-level resolution testing data to monitor for viral outbreaks in future epidemics/pandemics. It can be used for ongoing viral surveillance for bioterrorism activities or to detect the presence of future pandemic viruses as they reach new areas. It can be used to provide early data, even when the pathogen is unknown, of transmission risk in many congregate settings, such as nursing homes, prisons, schools, and childcare. The scientific findings support evidenced-based recommendations on ventilation upgrades to reduce disease transmission with direct human health data. Interventions can be non-pharmaceutical and do not require occupant action. These findings are applicable to influenza, RSV, adenovirus, and other respiratory pathogens. Overall, associations from this study lay the groundwork for widespread implementation to support the fight against a wide range of respiratory illnesses.

### REFERENCES

ADDIN Mendeley Bibliography CSL_BIBLIOGRAPHY 1. Dannemiller, K. C., Gent, J. F., Leaderer, B. P. & Peccia, J. Indoor microbial communities: Influence on asthma severity in atopic and nonatopic children. J. Allergy Clin. Immunol. 138, (2016). *PI had NSF GRFP as PhD student
2. Dannemiller, K. C. et al. Next-generation DNA sequencing reveals that low fungal diversity in house dust is associated with childhood asthma development. Indoor Air 24, n/a-n/a (2013). *PI had NSF GRFP as PhD student
3. Cochran, S. J. et al. Fungal diversity in homes and asthma morbidity among school-age children in New York City. Environ. Res. 239, 117296 (2023).
4. Low, S. Y., Dannemiller, K., Yao, M., Yamamoto, N. & Peccia, J. The allergenicity of Aspergillus fumigatus conidia is influenced by growth temperature. Fungal Biol. 115, (2011). *PI had NSF GRFP as PhD student
5. Yamamoto, N., Hospodsky, D., Dannemiller, K. C., Nazaroff, W. W. & Peccia, J. Indoor emissions as a primary source of airborne allergenic fungal particles in classrooms. Environ. Sci. Technol. 49, (2015). *PI had NSF GRFP as PhD student
6. Dannemiller, K. C., Gent, J. F., Leaderer, B. P. & Peccia, J. Influence of housing characteristics on bacterial and fungal communities in homes of asthmatic children. Indoor Air 26, (2016). *PI had NSF GRFP as PhD student
7. Dannemiller, K. C., Lang-Yona, N., Yamamoto, N., Rudich, Y. & Peccia, J. Combining real-time PCR and next-generation DNA sequencing to provide quantitative comparisons of fungal aerosol populations. Atmos. Environ. 84, 113-121 (2014). *PI had NSF GRFP as PhD student
8. Hegarty, B., Dannemiller, K. & Peccia, J. Gene expression of indoor fungal communities under damp building conditions: implications for human health. Indoor Air 0-2 (2018) doi: 10.1111/ina.12459.
9. Nastasi, N. et al. Morphology and quantification of fungal growth in residential dust and carpets. Build. Environ. 174, 106774 (2020).
10. Dannemiller, K. C. Moving towards a robust definition for a “healthy” indoor microbiome. mSystems 4, (2019).
11. Renninger, N. et al. Indoor Dust as a Matrix for Surveillance of COVID-19. mSystems 6, (2021).
12. Nastasi, N. et al. Persistence of viable MS2 and Phi6 bacteriophages on carpet and dust. Indoor Air 32, e12969 (2022). *Funded by NSF CAREER award
13. Green, B. J. et al. Airborne fungal fragments and allergenicity. Med. Mycol. 44 Suppl 1, 245-255 (2006).
15. Dannemiller, K. C., Weschler, C. J. & Peccia, J. Fungal and bacterial growth in floor dust at elevated relative humidity levels. Indoor Air 27, (2017).
16. Hegarty, B., Dannemiller, K. C. & Peccia, J. Metabolic activity of indoor fungi at variable water activities. Prep. (2018).
17. Haines, S. R., Siegel, J. A. & Dannemiller, K. C. Modeling microbial growth in carpet dust exposed to diurnal variations in relative humidity using the “Time-of-Wetness” framework. Indoor Air 30, 978-992 (2020).
18. Holcomb, D. A. & Stewart, J. R. Microbial Indicators of Fecal Pollution: Recent Progress and Challenges in Assessing Water Quality. Curr. Environ. Heal. Reports 7, 311-324 (2020).
19. Wu, Z., Greaves, J., Arp, L., Stone, D. & Bibby, K. Comparative fate of CrAssphage with culturable and molecular fecal pollution indicators during activated sludge wastewater treatment. Environ. Int. 136, 105452 (2020).
20. Stachler, E. et al. Quantitative CrAssphage PCR Assays for Human Fecal Pollution Measurement. Environ. Sci. Technol. 51, 9146-9154 (2017).

1. A method for determining concentration or presence of at least one microbe in a built environment, the method comprising a) collecting a sample; and b) measuring concentration of at least one indicator organism, wherein the concentration of the indicator organism correlates to the concentration or presence of the at least one microbe, and further wherein the indicator organism and the microbe are different organisms.

2. The method of claim 1, wherein the built environment comprises a building or vehicle.

3. The method of claim 1, wherein the concentration or presence in the built environment correlates to the concentration or presence of the microbe in a subject who has been present within the built environment.

4. The method of claim 1, wherein the sample is collected from a ventilation or exhaust system, HVAC system, dust collection system, vacuum, air purifier, or vent.

5. The method of claim 1, wherein the microbe comprises a virus, bacteria, or fungi.

6. The method of claim 1, wherein at least two microbes are quantified based on levels of the one indicator organism.

7. The method of claim 1, wherein at least one marker from the indicator organism is measured.

8. The method of claim 7, wherein the marker is a molecular marker.

9. The method of claim 8, wherein the marker is a nucleic acid, metabolite, small molecule, or protein.

10. The method of claim 1, wherein more than one indicator is measured.

11. The method of claim 8, wherein more than one molecular marker from the same indicator is measured.

12. The method of claim 1, wherein the indicator organism is a bacteriophage.

13. The method of claim 1, wherein the indicator organism is found in dust.

14. A device for determining concentration or presence of at least one microbe in a built environment, wherein the device comprises an input unit for receiving a sample, a means for measuring presence or concentration of an indicator organism within the sample, and an output unit for indicating the presence or concentration of the at least one indicator organism, wherein the indicator organism and the microbe are different organisms.

15. The device of claim 14, wherein the device comprises a computer means for calculating presence or concentration of the indicator organism(s).

16. The device of claim 15, wherein the computer means correlates the presence or concentration of the indicator organism(s) to the presence or correlation of the microbe.

17. The device of claim 14, wherein the output unit comprises a display.

18. (canceled)

19. (canceled)

20. The device of claim 14, wherein the sample is collected from a ventilation or exhaust system, HVAC system, dust collection system, vacuum, air purifier, or vent.

21. (canceled)

22. (canceled)

23. (canceled)

24. (canceled)

25. (canceled)

26. (canceled)

27. (canceled)

28. (canceled)

29. A kit for determining concentration or presence of at least one microbe in a built environment, the kit comprising a) means for collecting a sample; and b) means for measuring concentration of at least one indicator organism, wherein the concentration of the indicator organism correlates to the concentration or presence of the at least one microbe, and further wherein the indicator organism and the microbe are different organisms.
